(19) United States
(12) Patent Application Publication
(10) Pub. No.: US 2007/0105147 A1
(43) Pub. Date: May 10, 2007

(54) Rapid method for screening compounds for in vivo activity

(76) Inventor: Piotr Lassota, Succasunna, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/643,248

(22) Filed: Dec. 21, 2006

Related U.S. Application Data
(63) Continuation of application No. 10/250,739, filed on Jul. 7, 2003, now abandoned.
(60) Provisional application No. 60/260,571, filed on Jan. 9, 2001.
(60) Provisional application No. 60/302,614, filed on Jul. 2, 2001.

(51) Int. Cl. C12Q 1/68; C12Q 1/66

(52) U.S. Cl. 435/6; 435/8

(57) ABSTRACT

The present invention provides a rapid method for screening potentially pharmaceutically useful compounds for activity in vivo. The method has the steps of growing a target cell into which a reporter gene was introduced in a biocompatible, semipermeable encapsulation device; implanting the semi-permeable encapsulation device into a subject; administering a potentially pharmaceutically active compound to said subject; removing said encapsulation device from said subject after in vivo exposure to the potentially pharmaceutically active compound and evaluating said target cell for reaction to said potentially pharmaceutically active compound by measuring the expression of said reporter gene.

The present invention is directed to a method for screening compounds for pharmaceutical activity in an animal.

The desire for effective treatment against disease states, such as neoplastic growth, especially cancerous growth, has created a need for a quick and reliable way to screen potential chemotherapeutic agents. Different types of tumor cell lines tend to react differently to various chemotherapeutic agents, requiring a large number of experiments to screen one possible agent against various potential target cells and cell lines. Although in vitro screening processes using different cell lines are widely used to screen potential chemotherapeutic agents, the exposure of cells to drugs in vitro is highly artificial and does not reflect metabolic or systemic modification of the agents in the body. In addition, recent progress in understanding pathophysiological processes underlying diseases allows one to direct pharmacological intervention to well-defined targets (e.g., by modulation of expression, or activity of enzymes, or signal transduction molecules/pathways). Therefore, there is a need for rapid assays that would evaluate interactions of potential drug candidates with their targets in live animals. These “mechanistic” assays can potentially replace “symptomatic” models, where the readout is the progression of the disease and not an effect on the target of the chemotherapeutic intervention.

There are in vivo models with which potential chemotherapeutic agents are screened. These models involve implanting tumor cells into a laboratory animal, treating the animal with a possible chemotherapeutic agent, and then monitoring the animals to determine the effects of treatment on the tumor cells. Exemplary models include (1) the subcutaneous tumor model, in which live tumor cells are surgically implanted or tumor cell suspensions are injected under the skin of a laboratory animal; (2) orthotopic model where live tumor cells are surgically implanted or tumor cell suspensions are injected into the organ of tumor origin (i.e. prostate tumor cells into the prostate, lung tumor cells into the lungs or the subrenal tumor model, in which tumor cells are surgically implanted under the kidney capsule of laboratory animals); (3) the peritoneal model, in which the tumor cells are injected into the peritoneal cavity; and (4) the metastasis model, in which the tumor cells are directly injected into the blood vessels of a laboratory animal. The implanted tumor is then monitored to ensure that it is growing in the implanted animal. The resulting tumorous animal is then used to screen potential chemotherapeutic agents. Such in vivo models are labor intensive, and require a large number of test animals and a large amount of the tested compound. Furthermore, such in vivo models are time-consuming processes that require sufficient time for the implanted tumor to grow in the animal. It typically takes more than 4 to 5 weeks to obtain in vivo test results. Thus, there remains a need for an in vivo test method that can be utilized to rapidly screen compounds.

### SUMMARY

The present invention provides a rapid method for screening compounds for pharmaceutical activity. The method embraces the steps of (a) growing a target cell into which a reporter gene was introduced in a biocompatible, semi-permeable encapsulation device; (b) implanting the semi-permeable encapsulation device into a subject; (c) administering a potentially pharmaceutically active compound to said subject; (d) removing said encapsulation device from said subject after in vivo exposure to the potentially pharmaceutically active compound and (e) evaluating said target cell for reaction to said potentially pharmaceutically active compound by measuring the expression of said reporter gene.

The reporter gene may encode any product which is suitable for detection. Preferrably, the reporter gene encodes an easily assayed product which allows its detection in situ, such as for example β-galactosidase or preferably green fluorescent protein and especially luciferase.

Desirably, a reporter gene that produces a light-generating moiety, preferably a bioluminescent moiety, is introduced into the target cell in order to yield a target cell that is capable of emitting light. Evaluation of the target cell for reaction to the potentially pharmaceutically active compound can then be performed by simply measuring the intensity of light generated by the light-generating moiety.

In a first preferred aspect, the gene sequence for the bioluminescent moiety is operably-linked to a promoter that controls expression of a protein or enzyme that is associated with a physiological condition, for example, a protein or an enzyme that is underexpressed or preferably overexpressed as a result of the physiological condition. In this aspect, the expression level of the light-generating moiety is modulated in a manner predictive of the physiological condition.

In a second aspect, the gene sequence for the bioluminescent moiety is operably-linked to a constitutive promoter and the amount of the light-generating moiety present is proportional to the number of target cells present. Thus, there is less light emitted compared to a control if the compound being screened causes reduced proliferation or death in the cell line.

The method is highly advantageous over prior in vivo screen models. For example, the present method can be used to test antitumor drugs for in vivo efficacy with short turnover time. Unlike conventional xenograft models, which can take more than 4 to 5 weeks to obtain a result, the present method can produce results in less than 10 days. In particular a mechanistic reporter gene model, for example, the instance where a gene of interest is replaced by luciferase and light emission is a measure of the impact of a new drug candidate on the expression of that gene, can produce results in less than 4 days.

### BRIEF DESCRIPTION OF THE DRAWINGS

FIG. 1 is a graphic representation of the light emission by H1299C2 cells in hollow fibers retrieved from athymic, nude mice 24 hours after intravenous treatment with N-hydroxy-3-[4-[[(2-hydroxyethyl)[2-(1H-indol-3-yl)-ethyl]-amino]methyl]phenyl]-2E-2-propenamide (compound 1) and N-hydroxy-3-[4-[[[2-(2-methyl-1H-indol-3-yl)-ethyl]-amino]methyl]phenyl]-2E-2-propenamide (compound 2).

### DETAILED DESCRIPTION

The present invention provides a test method for quickly evaluating a compound for pharmaceutical activity in vivo. The present method additionally can be used to test the compound for tissue specificity and/or a delivery system containing the compound. Furthermore, the method can be used to detect the systemic localization of the test compound and/or a delivery system containing the compound in a subject, e.g., a test animal, which is preferably a mammal.

In a first preferred aspect, the present method uses one or more modified reporter genes associated with a target cell, for example, neoplastic cells, to evaluate the pharmaceutical activity of a compound. Unlike conventional methods for monitoring the level of protein or enzyme expressed by a reporter gene associated with a target cell, the present invention measures ex vivo for example the intensity of light generated by a light-generating moiety whose expression is operably-linked to a promoter that also controls the expression of a protein or enzyme the expression level of which is dependent on a physiological condition.

Thus, in accordance with the first aspect of the present invention, the expression of the light-generating moiety, or lack thereof, is predictive of the effect that the compound will have on the expression of the protein or enzyme of interest when the target cell is exposed to the compound. Usually, the protein or enzyme of interest is known to be overexpressed or underexpressed as a result of a physiological condition.

Since the expression of the light-generating moiety is accurately correlated with the intensity of the light produced by the target cell expressing the light-generating moiety, the expression of the light-generating moiety by the target cell is evaluated simply by measuring the intensity of light produced by the target cell. Since the same promoter controls the expression of both the protein or enzyme of interest and the light-generating moiety, the effect of the compound on the expression of the light-generating moiety also correlates with the effect that the compound is likely to have on the expression of the protein or enzyme of interest in the cell line.

In a second aspect, the ability of the test compound to kill or modulate the proliferation of a cell line is measured. In this second aspect, a reporter construct that contains e.g. a gene for producing a bioluminescent molecule operably-linked to a constitutive promoter is introduced into a target cell of interest. A constitutive promoter means that such a promoter is constitutively active in the target cell. Thus, the amount of the bioluminescent molecule present, and thus the intensity of the light generated, is related to the proliferation of the target cells, and the proliferation of the target cells, or the lack thereof, is evaluated by measuring the intensity of the light generated by the target cells.

The target cells can be selected from various cell lines, which include various mammalian cell lines, especially human cell lines. In the area of pharmaceutical cancer research, examples of suitable cell lines include cancerous and non-cancerous human tumor cell lines (e.g., melanomas, lung tumor lines, renal tumor lines, colon tumor lines, prostate tumor lines, ovarian tumor lines, breast tumor lines, central nervous system tumor lines, leukemic cell lines, etc.); human fibroblasts; human leukocytes; and murine tumor cell lines (e.g., P388 murine leukemia).

Light-generating moieties, especially bioluminescent molecules, and gene sequences thereof are known in the art and are available commercially. Especially useful light-generating moieties include the luciferase family (e.g. firefly luciferase, click beetle luciferases and their genetically modified variants) and acquorin family of bioluminescent molecules. Other useful light-generating moieties are also known, for example, various fluorescent protein bioluminescent molecules, e.g., green fluorescent protein, are disclosed in U.S. Pat. Nos. 5,625,048 and 5,804,387 and are commercially available.

Vector constructs of reporter genes encoding e.g. such bioluminescent molecules can be introduced into target cells, such as tumor cell lines, by means of any known method such as e.g. transfection or transduction. For example, it is known that luciferase vector constructs can be adapted for use in transfecting a variety of host cells, including most bacteria, and many eukaryotic cells (luc constructs).

The target cells are then placed in a semi-permeable encapsulation device that is made from a biocompatible material. The term “biocompatible” is used here to mean that the material by itself or in combination with living tissues does not produce foreign body reaction or fibrosis. As a preferred embodiment of the present invention, the semi-permeable encapsulation device is a permeable hollow fiber or dialysis tubing, which is produced from, for example, polysulfone (PS), polyvinylidene fluoride (PVDF), cellulose acetate (CA-E), saponified cellulose ester (SCE), polypropylene (PP), regenerated cellulose (RC) or cellulose ester (CE). Of these, particularly suitable is a hollow fiber produced from PVDF. Preferably, the encapsulation device has a dimension such that it does not interfere with normal activities of the subject animal when it is implanted, for example, a hollow fiber having internal diameter of from about 0.5 to 5 mm or 0.5 to 3 mm, most preferably about 1 mm. The encapsulation device is semi-permeable and is designed to allow diffusion of the test compounds into and out of the device, as well as nutrients and other necessary ingredients that are required to sustain the growth and survival of the cells grown in the device. Preferably, the semi-permeable device is made from a material that has a molecular weight cutoff of 50,000 Dalton or higher, more preferably 100,000 Dalton or higher. Particularly suitable semi-permeable device has a molecular weight cutoff between 50,000 Dalton and 500,000 Dalton.

A selected encapsulation device is prepared and loaded with the target cells. For example, the device, e.g., PVDF hollow fibers, is prepared by flushing with 70% ethanol solution and incubating in 70% ethanol at room temperature for a period of about 24 hours or longer. The alcohol is removed with a sterile water rinse. After appropriate preparation, the target cells are prepared and placed in the device. The target cells are prepared at a cell density appropriate to maintain cellular growth in the device. This density varies for each cell line, and must be established for each individually. As a general rule, most cells can be cultured at 1-10×10⁶ cells/mL of culture medium, although higher and lower ranges can be used. The target cells are transferred to sterile syringes and the encapsulation device is filled using a needle of the appropriate gauge to fit into the device. The end on the encapsulation device is then sealed, e.g., heat sealed or glue sealed using surgical glue.

The filled encapsulation device is implanted into laboratory animals, e.g., a mammal, including mice, rats and dogs. The filled device can be incubated in vitro to allow stabilization of the cell culture before it is implanted. Preferably, the device is implanted intraperitoneally or subcutaneously. Each laboratory animal can have more than one implant and have implants in more than one site. It is especially desirable to implant a number of the devices in various sites in a subject animal such that tissue specificity and localization of a pharmaceutically active compound administered to the animal can be monitored. The implanted device can be allowed to remain in the host animal for an extended period since the biocompatible encapsulation device does not interfere with the animal's defensive system.

After implantation, the laboratory animal is dosed under an appropriate regimen with the compound being evaluated. After permitting the compound to interact with the implanted target cell for an appropriate period of time, the filled encapsulation devices are removed from the animal. The expression of the reporter gene by the target cells is measured depending on the reporter gene used, e.g. by measuring the luminescence, and preferably compared with a control that was not exposed to the compound being evaluated.

The luminescence of an expressed light-generating moiety can be detected by various means, including a CCD camera or IVIS camera, and analyzed. Although the image of the light-generating biological activities can be processed in vivo, i.e., obtaining the image through the skin of the animal, according to the present invention it is more desirable to remove the encapsulation device from the animal prior to measuring the light emission. This ex vivo measurement of the light emission permits simpler and more accurate processing of the encapsulation device to obtain the image. Indeed, the ex vivo measurement of the light emitted by the biological entity encapsulated in hollow fibers provides a significantly more acceptable signal to noise ratio, in particular, by permitting the biological entity to be exposed to saturating concentrations of factors required for light emission, such as ATP, oxygen and/or substrate, and by reducing the influence of other factors that might influence light emission in vivo. For example, when luciferase is utilized as the light-generating moiety, attempts to image the device in vivo may result in unacceptably low signal due to the fact that the concentration of luciferin cannot be increased to a sufficient level within the animal to obtain high intensity illumination since luciferin is toxic at high concentrations.

The present method can be utilized as a first line screen to test potential drug candidates for in vivo efficacy. The major advantages of the method include short turnover time (3-8 times shorter compared the s.c. xenograft model), high throughput (4 times higher than for the s.c. xenograft model), greatly reduced need for animals, and the ability to test 4 different tumors in one animal. It has been found that 5-fluorouracil, paclitaxel, vincristine, mitoxantrone, doxorubicin, etoposide, camptothecin, cis-platinum, and mitomycin C, which represent a wide variety of clinically validated mechanisms of action, produce significant activity when tested on various tumor cell lines with the present method. It has also been found that tumor cells in the encapsulation device responded to antitumor agents delivered through all clinically relevant routes of administration: intravenously, orally, and intraperitoneally.

The following examples illustrate aspects of this invention. They are intended to describe, but not limit, the invention.

### EXAMPLE 1

A colon carcinoma cell line (PC-3M available from MD Anderson Cancer Center, Houston, Tex.) is transfected with a vector containing the gene for luciferase (luc gene) under the control of a constitutive promoter (pGL3 vector; Promega). The transfected cell line is propagated, and expanded in RPMI 1640 medium containing 10% heat-inactivated Fetal Bovine Serum (BRL Life Technologies, Grand Island, N.Y.), Cell expansions for implantations are done in T-75 tissue culture flasks (Costar, Corning, N.Y.). All cell cultures are performed in an incubator, at 37° C. with a humidified atmosphere, containing 5% CO₂. Cells are harvested at 70-90% confluency using 0.25% Trypsin-EDTA solution (BRL Life Technologies, Grand Island, N.Y.). After trypsinization cells are diluted in media to a desired concentration and kept at 37° C. in the incubator. 20 minutes prior to use the cells are vortexed for two seconds and placed on ice. Cell suspensions for injection into the encapsulation device is made at a concentration of 1×10⁶ cells/mL.

The transfected tumor cells are placed in a semi-permeable encapsulation device, e.g., a hollow fiber having an inner diameter of about 1 mm, and grown in the device before the encapsulation device is implanted in a subject. For example, 12 μl of the above-described colon carcinoma cell suspension is placed in a PVDF hollow fiber (available from Spectrum, Gardena, Calif. and having 1.0 mm internal diameter and 500,000 molecular weight cutoff), and the opening is heat sealed, making a sealed tube. The tube is placed in the RPMI 1640 medium containing 10% heat-inactivated Fetal Bovine Serum. Ten outbred athymic (nu/nu) female mice (“Crl:NU/NU-nuBR” from Charles River Laboratories, Wilmington, Mass.), are anesthesized at one time by i.p. administration of 0.2 mL of a 7:3 mixture of 100 mg/mL Ketamine and 20 mg/mL Xylazine, diluted 1:5 with 0.9% saline (McGaw Inc., Irvine, Calif.). Skin at both incision sites is disinfected with Novalsan (Henry Schein, Port Washington, N.Y.). After the incision is made with surgical scissors in the skin at the nape of the neck, two tubes are implanted through it, into each side of the animal using an 11 gauge trocar (Popper & Sons, Fischer Scientific, Pittsburgh, Pa.). One wound clip (Clay Adams, Becton Dickinson, Franklin Lakes, N.J.) is used to close the skin. For the intraperitoneal implantation an approximately 5 mm incision is made in the skin, dorsoventrally, just to the tail side of the spleen. The incision is followed by an incision in the thin layer of adipose tissue, and then a smaller incision (2 mm) in the now exposed peritoneum. Inserting each fiber requires sliding it ⅓ of the way straight into the perforation, moving the outer end of each fiber towards the front of an animal, and sliding the fibers back towards the tail. Two skin staples are used to close the skin. After the surgery is completed each animal receives one subcutaneous dose of 0.1 mL Butorphenol (from Henry Schein, Port Washington, N.Y., diluted to 0.02 mg/mL with 0.9% saline). Animals are allowed to recover from the anaesthesia on a heating pad, before returning to their cages.

The animals with the implanted device are dosed with test pharmaceutical compounds. The compounds are administered to the animals by various means including intravenous, intraperitoneal, subcutaneous, oral and/or percutaneous routes on any number of schedules. The treated animals are grown under laboratory conditions to allow the system to process and deliver the compound within the body. After allowing the compound to have enough time to interact with the implanted target cells, the efficacy of the compound is accessed by measuring the intensity of light produced by the light-generating moiety expressed by the cells. When the test compound has an inhibitory affect on the cells, the expressed level of the light-generating moiety, and thus the light intensity, is diminished compared to the control animal that was implanted with the device but did not receive the test compound. Conversely, when the test compound has a stimulatory affect on the cells, the expressed level of the light-generating moiety is increased compared to the control animal. Similarly, when the compound does not provide any interaction with the target cells or it does not reach a specific site or tissue of the test animal, the intensity of luminescence is not different from that of the control animal.

### EXAMPLE 2

A human lung cancer cell line, H1299, is obtained from the American Type Culture Collection, ATCC, Rockville, Md.) and cultured in the RPMI 1640 medium supplemented with 10% heat-inactivated Fetal Bovine Serum (Life Technologies, Grand Island, N.Y.). Cells harvested at approx. 85% confluency are washed with ice cold phosphate buffered saline (PBS) and suspended in the ice cold PBS at a concentration of 2×10⁷ cells/mL. One half of one mL of the cell suspension is used for transfection with 10 μg of p21^{WAF1/Cip1} luciferase plasmid and NeotetR selectable marker. Transfection is performed by electroporation at 0.3 V and 500 F. Cells are then plated in the above medium containing 0.4 mg/mL G-418 (Life Technologies, Grand Island, N.Y.). One colony is selected, diluted to one cell/well and a single clone is expanded to generate p21^{WAF1/Cip1} promoter-luciferase stable cell line designated H1299C2. The cell line tests negative for Mycoplasma contamination (Rapid Detection System by Gen-Probe, Inc., San Diego, Calif.) and for Mouse Antibody Production (MA BioServices, Inc., Rockville, Md.).

The H1299C2 cell line is propagated and expanded for implantation in RPMI 1640 medium containing 10% heat-inactivated Fetal Bovine Serum (Life Technologies, Grand Island, N.Y.) in a cell culture incubator, at 37° C. with humidified atmosphere, containing 5% CO₂. Cells are harvested at 70-90% confluency using 0.25% Trypsin-EDTA solution (Life Technologies, Grand Island, N.Y.). After trypsinization cells are diluted with the above medium to a concentration of 1×10⁶ cells/mL, vortexed for two seconds and placed on ice until injection onto a hollow fiber.

PVDF hollow fibers (Spectrum, Gardena, Calif.) are soaked in 70% Ethanol for a minimum of 72 hours prior to use. All subsequent handling of the hollow fibers is done under a laminar flow hood using aseptic procedures. Individual hollow fibers are removed from the pan and flushed on the work surface with 3-4 mL of the ice-cold media using a 20 mL syringe with a 20-gauge needle. The H1299C2 cell suspension is then slowly injected into the Hollow Fiber using a 3-mL syringe equipped with a 20-gauge needle. Both ends of the fiber are subsequently sealed using a flat needle holder, heated in a bacteriological incinerator (Fischer Scientific, Pittsburgh, Pa.). Using the hollow fiber heat-sealing machine set at 113° C. (model FS-2 Mark 1, Outsource 2000, Huntsville, Ala.), the entire length of the hollow fiber was then sealed into 1.5 cm microcapsules (hereinafter referred to as “Fibers”), with each Fiber containing about 12 μl (12,000 cells) of the cell suspension. Fibers are then placed into 6 well plates (12 Fibers per well containing 5 mL of the media) and plates were incubated overnight in the incubator at 37° C.

Plates containing Fibers prepared as described above are placed under the laminar hood on top of the heating pad. Ten outbred athymic (nu/nu) female mice (“Crl:NU/NU-nuBR” from Charles River Laboratories, Wilmington, Mass.), are anesthesized at one time by intraperitoneal administration of 0.2 mL of a 7:3 mixture of 100 mg/mL Ketamine and 20 mg/mL Xylazine, diluted 1:5 with 0.9% saline (McGaw Inc., Irvine, Calif.). Skin at the incision site is disinfected with Novalsan (Henry Schein, Port Washington, N.Y.). After an incision is made with surgical scissors in the skin at the nape of the neck, one Fiber is implanted through it, using an 11 gauge trocar (Popper & Sons, Fischer Scientific, Pittsburgh, Pa.). One wound clip (Clay Adams, Becton Dickinson, Franklin Lakes, N.J.) is used to close the skin. After the implantation is completed each animal receives one subcutaneous dose of 0.1 mL Butorphenol (from Henry Schein, Port Washington, N.Y., diluted to 0.02 mg/mL with 0.9% saline). Animals are allowed to recover from the anesthesia on a heating pad, before returning to their cages.

The animals are treated 24 hours after the implantation of the Fibers with a single, intravenous dose (50, 25 or 12.5 mg/kg) of compound 1 or 2 (for their preparation see below), both histone deacetylase inhibitors. For the intravenous dosing the compounds are formulated as a solution in 5% Dextrose in water (vehicle) for injection.

All animals are sacrificed 24 hours after the dosing, after which the Fibers are retrieved and placed in six well plates (one fiber/well) containing 2.5 mL of media with 0.25 mg/mL of D-luciferin (Xenogen Corporation, Alameda, Calif.). After 15 minutes of incubation, the light emission is recorded using a Hamamatsu CCD camera (Xenogen Corporation, Alameda, Calif.). The images are subsequently analyzed for the light output using the LivingImage™ v.2.10 software (Xenogen Corporation, Alameda, Calif.).

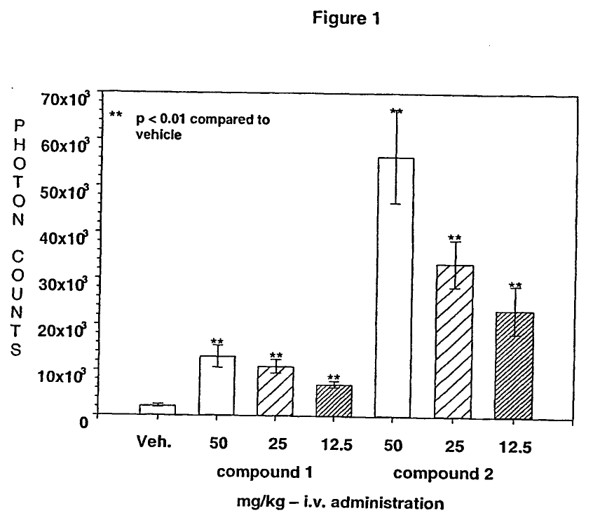

Published results indicate that inhibition of histone deacetylation results in activation of p21^{WAF1/Cip1} promoter and thus in expression of the p21^{WAF1/Cip1} tumor suppressor protein. In this example, a reporter gene is used wherein p21^{WAF1/Cip1} gene is replaced by the firefly luciferase gene and activation of p21^{WAF1/Cip1} promoter produces luciferase. The histone deactelylase inhibitors cause statistically significant (p<0.01, one tailed Student t-test) and reproducible expression of the reporter gene (firefly luciferase) in vivo as judged by registered light emission from the Fibers retrieved from animals dosed with the compounds 1 or 2 (see FIG. 1).

Preparation of Compound 1:

A solution of 3-(4-{[2-(1H-indol-3-yl)-ethylamino]-methyl}-phenyl)-(2E)-2-propenoic acid methyl ester (12.6 g, 37.7 mmol), (2-bromoethoxy)-tert-butyldimethylsilane (12.8 g, 53.6 mmol), (i-Pr)₂NEt, (7.42 g, 57.4 mmol) in dimethylsulfoxide (100 mL) is heated to 50° C. After 8 hours the mixture is partitioned with CH₂Cl₂/H₂O. The organic layer is dried (Na₂SO₄) and evaporated. The residue is chromatographed on silica gel to produce 3-[4-({[2-(tert-butyldimethylsilanyloxy)-ethyl]-[2-(1H-indol-3-yl)-ethyl]-amino}-methyl)-phenyl]-(2E)-2-propenoic acid methyl ester (13.1 g). A solution of KOH (12.9 g 87%, 0.2 mol) in methanol (MeOH) (100 mL) is added to a solution of HONH₂—HCl (13.9 g, 0.2 mol) in MeOH (200 mL) and a precipitate results. After 15 minutes the mixture is filtered, the filter cake washed with MeOH and the filtrate evaporated under vacuum to approximately 75 mL. The mixture is filtered and the volume adjusted to 100 mL with MeOH. The resulting solution 2M HONH₂ is stored under N₂ at −20° C. for up to 2 weeks. Then 3-[4-({[2-(tert-butyldimethylsilanyloxy)-ethyl]-[2-(1H-indol-3-yl)-ethyl]-amino}-methyl)-phenyl]-(2E)-2-propenoic acid methyl ester (6.50 mmol) is added to 2 M HONH₂ in MeOH (30 mL, 60 mmol) followed by a solution of KOH (420 mg, 6.5 mmol) in MeOH (5 mL). After 2 hours dry ice is added to the reaction and the mixture is evaporated to dryness. The residue is dissolved in hot MeOH (20 mL), cooled and stored at −20° C. overnight. The resulting suspension is filtered, the solids washed with ice cold MeOH and dried under vacuum, producing N-hydroxy-3-[4-({[2-(tert-butyldimethylsilanyloxy)-ethyl]-[2-(1H-indol-3-yl)-ethyl]-amino}-methyl)-phenyl]-(2E)-2-propenamide. The hydroxamic acid (5.0 g, 13.3 mmol) is then dissolved in 95% trifluoroacetic acid/H₂O (59 mL) and heated to 40-50° C. for 4 hours. The mixture is evaporated and the residue purified by reverse phase HPLC to produce N-hydroxy-3-[4-[[(2-hydroxyethyl)[2-(1H-indol-3-yl)-ethyl]-amino]methyl]phenyl]-2E-2-propenamide as the trifluoroacetate salt (m/z 380 [MH⁺]), which can then be converted into the free base.

Preparation of Compound 2:

A suspension of LiAlH₄ (17 g, 445 mmol) in dry tetrahydrofuran (1000 mL) is cooled to 0° C. and 2-methylindole-3-glyoxylamide (30 g, 148 mmol) is added in portions over 30 min. The mixture is stirred at room temperature for 30 min and then maintained at reflux for 3 hours. The reaction is cooled to 0° C. and treated with H₂O (17 mL), 15% NaOH (aq., 17 mL) and H₂O (51 mL). The mixture is treated with MgSO₄, filtered and the filtrate evaporated to give 2-methyltryptamine which is dissolved in MeOH. Methyl 4-formylcinnamate (16.9 g, 88.8 mmol) is added to the solution, followed by NaBH₃CN (8.4 g) and acetic acid (1 equivalent). After 1 hour the reaction is diluted with NaHCO₃ (aq.) and extracted with ethyl acetate. The organic extracts are dried (MgSO₄), filtered and evaporated. The residue is purified by chromatography to give 3-(4-{[2-(2-methyl-1H-indol-3-yl)-ethylamino]-methyl}-phenyl)-(2E)-2-propenoic acid methyl ester. The ester is dissolved in MeOH, 1.0 M HCl/dioxane (1-1.5 equivalents) is added followed by diethyl ether (Et₂O). The resulting precipitate is filtered and the solid washed with Et₂O and dried thoroughly to give 3-(4-{[2-(2-methyl-1H-indol-3-yl)-ethylamino]-methyl}-phenyl)-(2E)-2-propenoic acid methyl ester hydrochloride. 1.0 M NaOH (aq., 85 mL) is added to an ice cold solution of the methyl ester hydrochloride (14.9 g, 38.6 mmol) and HONH₂ (50% aq. solution, 24.0 mL, ca. 391.2 mmol). After 6 hours, the ice cold solution is diluted with H₂O and NH₄Cl (aq., 0.86 M, 100 mL). The resulting precipitate is filtered, washed with H₂O and dried to afford N-hydroxy-3-[4-[[[2-(2-methyl-1H-indol-3-yl)-ethyl]-amino]methyl]phenyl]-2E-2-propenamide (m/z 350 [MH⁺]).

1. A method of screening a compound for pharmaceutical activity, comprising the steps of: (a) growing a target cell into which a report gene was introduced in a biocompatible, semi-permeable encapsulation device; (b) implanting the semi-permeable encapsulation device into a subject; (c) administering a potentially pharmaceutically active compound to said subject; (d) removing said encapsulation device from said subject after in vivo exposure to said compound and (e) evaluating said target cell for reaction to said compound by measuring the expression of said reporter gene.

2. The method of claim 1 wherein the reporter gene produces a light-generating moiety and evaluation of the target cell for reaction to the compound is done by measuring the intensity of light generated by the light-generating moiety.

3. The method of claim 2 wherein the reporter gene produces a bioluminescent moiety.

4. The method of claim 1 wherein the semi-permeable encapsulation device has a molecular cutoff of a least 50,000 Dalton.

5. The method of claim 1 wherein the target cell is selected from the group consisting of cancerous and non-cancerous human tumor cell lines; human fibroblasts; human leukocytes; and murine tumor cell lines.

6. The method of claim 1 wherein the target cell is a tumor cell.

7. The method of claim 6 wherein the target cell is a human tumor cell line selected from the group consisting of a melanoma cell line, a lung tumor cell line, a renal tumor cell line, a colon tumor cell line, a prostate tumor cell line, an ovarian tumor cell line, a breast tumor cell line, a central nervous system tumor cell line and a leukemic cell line.

8. The method of claim 1 wherein the reporter gene contains a promoter that also controls expression of a protein or enzyme that is associated with a physiological condition.

9. The method of claim 8 wherein said protein or said enzyme is overexpressed by the target cell.

10. The method of claim 8 wherein said protein or said enzyme is underexpressed by the target cell.

11. The method of claim 8 wherein the reporter gene encodes luciferase.

12. The method of claim 8 wherein the target cell comprises a gene sequence encoding luciferase which is operably-linked to a p21^{WAF1/Cip1} promoter.

13. The method of claim 11 wherein the target cells are evaluated after being exposed to a saturating amount of luciferin.

14. The method of claim 8 wherein the reporter gene encodes green fluorescent protein.

15. The method of claim 1 wherein the reporter gene contains a constitutive promoter.

16. The method of claim 15 wherein the reporter gene encodes luciferase.

17. The method of claim 16 wherein the target cells are evaluated after being exposed to a saturating amount of luciferin.
